(19) United States
(12) Patent Application Publication
(10) Pub. No.: US 2022/0125710 A1
(43) Pub. Date: Apr. 28, 2022

(54) COSMETIC COMPOSITION FOR SKIN BARRIER STRENGTHENING OR MOISTURIZING COMPRISING FERMENTED RICE EXTRACTS, PINEAPPLE EXTRACTS, AND GLYCERYL GLUCOSIDE MIXTURE

(71) Applicant: SKIN REVERSE LAB, Seoul (KR)

(72) Inventors: Seonkyo IN, Daejeon (KR); Jaehyun KIM, Anyang-si (KR)

(73) Assignee: SKIN REVERSE LAB, Seoul (KR)

(21) Appl. No.: 17/081,827

(22) Filed: Oct. 27, 2020

(51) Int. Cl. A61K 8/9794; A61K 8/60; A61K 8/02; A61K 8/04; A61Q 19/00

(52) U.S. Cl. CPC A61K 8/9794; A61K 8/602; A61Q 19/00; A61K 8/04; A61K 8/0212

(57) ABSTRACT

The present invention relates to a cosmetic composition and/or a skin external application agent for skin barrier strengthening or moisturizing comprising fermented rice extracts, pineapple extracts and glyceryl glucoside mixture.

### TECHNICAL FIELD

The present invention relates to a cosmetic composition and/or a skin external application agent for skin barrier strengthening or moisturizing comprising fermented rice extracts, pineapple extracts and glyceryl glucoside mixture.

### BACKGROUND

The skin performs a very important role as a barrier function to protect the individual from the outside. The barrier function is to protect against various external stimuli (chemical substances, air pollutants, dry environment, ultraviolet rays, etc.) and to prevent excessive dissipation of moisture in the body through the skin. This protective function may be maintained only when stratum corneum composed of keratinocytes is normally formed.

Among the epidermis, the outermost stratum corneum is formed from keratinocytes, and is composed of corneocytes with complete differentiation and a lipid layer surrounding them. The corneocytes are characteristic cells in which basal cells that continuously proliferate in the lowermost layer of the epidermis rise to the surface of the skin through a step-by-step change in shape and function. After a certain period has elapsed, old corneocytes are eliminated from the skin and new corneocytes take over their function. This repetitive sequence of changes is called “epidermal cell differentiation” or “keratinization”. During the keratinization process, the keratinocytes form the stratum corneum while producing a natural moisturizing factor (NMF) and intercellular lipids (ceramide, cholesterol, fatty acid), making the stratum corneum firm and flexible, thereby retaining the function as a skin barrier. When the stratum corneum becomes thinner and weaker, the structures of keratin and cell thin lipid membranes that are tightly bonded like bricks and cements become loose and cracks appear. Eventually, moisture evaporates and becomes dry, and the defense function against harmful substances is weakened.

Such a stratum corneum may easily lose its function due to lifestyle factors such as excessive washing or bathing, environmental factors such as dryness or air pollutants, or endogenous diseases such as atopic skin or senile skin. In fact, due to various factors that have increased since the modern age, the number of people complaining of dry skin symptoms and various disorders caused by them is increasing. Therefore, in order to maintain proper skin moisture, various studies have been conducted to supply moisture from the outside or to prevent moisture loss from the body, and various moisturizing agents having moisture retention capabilities have been developed. For example, the skin moisturizing agents is common to use a lipid component such as ceramide and a substance capable of increasing water retention in the stratum corneum, such as essential fatty acids and lipid complexes.

However, in recent years, harmful factors to the skin are gradually increased; the rate of stratum corneum generation and dropout is slowed due to changes in dietary habits, and the amount of moisturizing factors and lipids of the stratum corneum decreases due to the decline in the function of keratinocytes. Thus, there is a growing trend of people with skin where the stratum corneum does not function as a normal skin barrier. The breakdown of the function of the skin barrier causes various skin diseases such as dry skin, atopic dermatitis, contact dermatitis, and psoriasis. In order to cure such diseases, it is necessary to develop moisturizing agent superior in skin barrier strengthening or moisturizing improvement compared to conventional skin moisturizing agents.

### PATENT LITERATURE

Korean Patent Registration No. 10-1972531 (Apr. 19, 2019)

### SUMMARY

The present inventors have tried to develop a new material that is safe and superior in skin barrier strengthening or moisturizing improvement compared to conventionally known moisturizing agents. As a result, after confirming that the composition containing fermented rice extracts, pineapple extracts, and glyceryl glucoside mixture is effective in skin barrier strengthening or function improving, the present invention has been completed.

An object of the present invention is to provide a cosmetic composition and/or a skin external application agent for improving skin barrier strengthening or moisturizing, comprising a composition comprising fermented rice extracts, pineapple extracts and glyceryl glucoside mixture.

In addition, an another object of the present invention is to provide a cosmetic method for controlling a condition of a skin of a mammal by improving skin barrier strengthening or moisturizing except for treatment, using a composition comprising fermented rice extracts, pineapple extracts and glyceryl glucoside mixture,

However, the subject of the present invention as described above is exemplary, and thus the scope of the present invention is not limited thereto. Further, other objects and advantages of the present invention will become more apparent by the following detailed description, claims, and drawings.

According to the invention, the composition containing fermented rice extracts, pineapple extracts and glyceryl glucoside mixture may improve objective indicators related to skin barrier strengthening, e.g., water reduction indicators of the skin stratum corneum such as transepidermal water loss (TEWL), and increase skin moisture index, and skin barrier index may be strengthened. Therefore, according to the present invention, the composition for improving skin barrier strengthening or moisturizing may be expected to be useful as a cosmetic composition and/or skin external application agent for improving skin barrier strengthening or moisturizing.

### DETAILED DESCRIPTION

Hereinafter, according to a specific embodiment of the present invention, a cosmetic composition and/or skin external application agent for improving skin barrier strengthening or moisturizing will be described in detail. However, it is apparent to those skilled in the art that this embodiment is provided as an example of the invention, whereby the scope of the invention is not limited, and various modifications to the embodiments are possible within the scope of the invention.

As used herein, the term “skin barrier strengthening” means protecting, strengthening, or improving skin barrier function of the skin stratum corneum, and does not mean regeneration or function improvement of dermis or subcutaneous fat tissue. For example, the “skin barrier strengthening” prevents moisture loss in the stratum corneum, improves indicators of moisture reduction in the stratum corneum, such as transepidermal water loss (TEWL), and improves skin barrier indicators such as increasing skin moisture content (skin hydration). Accordingly, it is meant to bring about the protection, strengthening and/or improvement of the skin barrier function of the skin stratum corneum in the true sense.

As used herein, the term “skin moisturizing” means maintaining the homeostasis of a living body by appropriately controlling the moisture loss (moisture evaporation) of the skin.

In a first embodiment,

the present invention is intended to provide a cosmetic composition and/or skin external application agent for improving skin barrier strengthening or moisturizing, comprising a composition comprising fermented rice extracts, pineapple extracts and glyceryl glucoside mixture.

According to the cosmetic composition and/or skin external application agent of the present invention, the weight ratio of the fermented rice extracts, pineapple extracts and glyceryl glucoside mixture may be 1:1:1.

According to the cosmetic composition and/or skin external application agent of the present invention, the fermented rice extracts may contain antioxidants (Thioredoxin) having a concentration of 400 ug/L or more. The rice may be selected from the group consisting of brown rice and white rice. The fermentation may be carried out using natural yeast.

According to the cosmetic composition and/or skin external application agent of the present invention, the pineapple extracts may contain a ceramide content of 40 g/L or more.

According to the cosmetic composition and/or skin external application agent of the present invention, the glyceryl glucoside mixture may contain a content of a compound of glycerin and glucose of 60% or more having a purity of 90% or more after chemical bonding between glycerin and glucose.

According to the cosmetic composition and/or skin external application agent of the present invention, the cosmetic composition and/or skin external application agent may be appropriately blended with antioxidants, oily ingredients, ultraviolet absorbers, emulsifiers, surfactants, thickeners, alcohols, powder components, coloring materials, aqueous components, water and various skin nutrients, as components commonly used in cosmetics or skin external application agents as needed, within the range not impairing the effects of the present invention.

According to the present invention, the cosmetic composition and/or skin external application agent may be applied to at least one type selected from the group consisting of patch, mask pack, mask sheet, cream, tonic, ointment, suspension, turbid fluid, paste, lotion, gel, oil, pack, spray, aerosol, mist, foundation, powder, and oil paper. For example, the cosmetic composition and/or skin external application agent may be formulated with by a patch, mask pack, mask sheet, softening lotion, nourishing lotion, astringent lotion, nourishing cream, massage cream, eye cream, cleansing cream, essence, eye essence, cleansing lotion, cleansing foam, cleansing water, sunscreen, lipstick, soap, shampoo, surfactant-containing cleansing, bathing agent, body lotion, body cream, body oil, body essence, body cleanser, hair dye, hair tonic, etc., but is not limited thereto. The cosmetic composition and/or skin external application agent may be applied or deposited on at least one surface of the patch, mask pack, or mask sheet.

In a second embodiment,

the present invention is intended to provide a cosmetic method for controlling a condition of a skin of a mammal by improving skin barrier strengthening or moisturizing of the stratum corneum except for treatment, using a composition comprising fermented rice extracts, pineapple extracts and glyceryl glucoside mixture.

According to the cosmetic method for controlling a condition of a skin of a mammal of the present invention, the cosmetic method may include (a) directly applying the cosmetic composition for improving skin barrier strengthening or moisturizing to the skin of the mammal, or (b) contacting or attaching the applied or deposited patch, mask pack, or mask sheet with the cosmetic composition for improving skin barrier strengthening or moisturizing to the skin of the mammal, or sequentially proceeding the steps (a) and (b). In the step (a), the composition for improving skin barrier strengthening or moisturizing may be used in the lotion or cream.

According to the cosmetic method for controlling a condition of a skin of a mammal of the present invention, the cosmetic method may further comprise (c) removing the patch, mask pack or mask sheet from the skin of the mammal after step (b), and applying the composition for improving skin barrier strengthening or moisturizing to the skin of the mammal. In the step (c), the composition for improving skin barrier strengthening or moisturizing may be used in the lotion or cream.

Hereinafter, the present invention will be described in more detail through examples. However, these are only for describing the present invention in more detail, and the scope of the present invention is not limited thereto.

### EXAMPLE

### Example 1: Preparation Fermented Rice Extracts, Pineapple Extracts and Glyceryl Glucoside Mixture

The rice fermented extracts, eco-friendly pineapple extracts, and glyceryl glucoside compound were mixed in purified water containing 2% 1, 2-hexanediol, respectively, in a weight ratio according to Table 1 below. Here, the fermented rice extracts were induced to contain an antioxidant (Thioredoxin) concentration of 400 ug/L or more during the fermentation process of rice using natural yeast. The eco-friendly pineapple extracts were extracted so that the content of natural sugar ceramide could be 40 g/L or more by using an outer skin discarded after removing flesh. In the glyceryl glucoside compound, the content of glycerin & glucose compounds of a purity of 90% or more was found to be 60% or more after chemical bonding between the glycerin and the glucose. Each mixture was put into a water jacket reactor capable of maintaining a temperature, heated to 55 to 65° C., and stirred at 700 rpm for 4 hours. After the stirring was completed, the mixture was firstly filtered through a 100 um Cellulose Acetate filter, and secondly filtered through a 0.22 μm PVDF filter.

TABLE 1

| | Fermented rice | Pineapple | Glyceryl |
|---|---|---|---|
| | extracts | extracts | Glucoside |
| Example 1 | 0 | 1 | 1 |
| Example 2 | 1 | 0 | 1 |
| Example 3 | 1 | 1 | 0 |
| Example 4 | 1 | 1 | 1 |

### Experimental Example

### Experimental Example 1: Confirmation of Transepidermal Water Loss (TEWL) Alleviation Effect of the Composition According to the Present Invention

To induce an increase in TEWL, ethanol was applied to the inner arm of 10 men and women aged 26 to 44 years old, and then TEWL index of the corresponding site was measured using a skin condition-measuring device (GPSKIN) at five points. The purified water used as Examples 1 to 4 and control group was applied to the five points, and after 30 minutes, the TEWL indices were measured at the five points using the GPSKIN.

TABLE 2

| ID | Gender | Age | Sort | TEWL |
|---|---|---|---|---|
| 1 | M | 41 | Purified water | 13 |
| | | | Example 1 | 11 |
| | | | Example 2 | 12 |
| | | | Example 3 | 12 |
| | | | Example 4 | 9 |
| | F | 44 | Purified water | 14 |
| | | | Example 1 | 13 |
| | | | Example 2 | 12 |
| | | | Example 3 | 14 |
| | | | Example 4 | 8 |
| | F | 26 | Purified water | 10 |
| | | | Example 1 | 9 |
| | | | Example 2 | 9 |
| | | | Example 3 | 7 |
| | | | Example 4 | 6 |
| | F | 27 | Purified water | 11 |
| | | | Example 1 | 10 |
| | | | Example 2 | 10 |
| | | | Example 3 | 11 |
| | | | Example 4 | 6 |
| | F | 26 | Purified water | 14 |
| | | | Example 1 | 12 |
| | | | Example 2 | 12 |
| | | | Example 3 | 13 |
| | | | Example 4 | 9 |
| 6 | F | 28 | Purified water | 13 |
| | | | Example 1 | 11 |
| | | | Example 2 | 11 |
| | | | Example 3 | 12 |
| | | | Example 4 | 9 |
| 7 | F | 32 | Purified water | 24 |
| | | | Example 1 | 20 |
| | | | Example 2 | 19 |
| | | | Example 3 | 22 |
| | | | Example 4 | 15 |
| 8 | M | 30 | Purified water | 11 |
| | | | Example 1 | 9 |
| | | | Example 2 | 10 |
| | | | Example 3 | 10 |
| | | | Example 4 | 7 |
| 9 | M | 38 | Purified water | 12 |
| | | | Example 1 | 11 |
| | | | Example 2 | 11 |
| | | | Example 3 | 12 |
| | | | Example 4 | 9 |
| 10 | F | 45 | Purified water | 13 |
| | | | Example 1 | 10 |
| | | | Example 2 | 11 |
| | | | Example 3 | 11 |
| | | | Example 4 | 8 |

As a result, as may be seen from Table 2, Examples 1 to 4 exhibited reduced TEWL compared to the control group (purified water), and among them, an effect of the TEWL relaxation according to Example 4 was remarkably excellent.

### Experimental Example 2: Confirmation of an Effect of Skin Moisture Index of the Composition According to the Present Invention

In order to know the change in the moisture index of 10 testers, the moisture index was measured before applying the five points on the inside of the arm. The purified water used as Examples 1 to 4 and the control group was applied to the five points, and after 30 minutes, the moisture index was measured at the five points using the GPSKIN.

TABLE 3

| | | | | Moisture |
|---|---|---|---|---|
| ID | Gender | Age | Sort | index |
| 1 | M | 41 | Purified water | 52 |
| | | | Example 1 | 84 |
| | | | Example 2 | 85 |
| | | | Example 3 | 83 |
| | | | Example 4 | 96 |
| 2 | F | 44 | Purified water | 71 |
| | | | Example 1 | 88 |
| | | | Example 2 | 86 |
| | | | Example 3 | 85 |
| | | | Example 4 | 94 |
| 3 | F | 26 | Purified water | 54 |
| | | | Example 1 | 85 |
| | | | Example 2 | 83 |
| | | | Example 3 | 84 |
| | | | Example 4 | 91 |
| 4 | F | 27 | Purified water | 51 |
| | | | Example 1 | 82 |
| | | | Example 2 | 80 |
| | | | Example 3 | 81 |
| | | | Example 4 | 93 |
| 5 | F | 26 | Purified water | 53 |
| | | | Example 1 | 91 |
| | | | Example 2 | 89 |
| | | | Example 3 | 86 |
| | | | Example 4 | 96 |
| 6 | F | 28 | Purified water | 54 |
| | | | Example 1 | 90 |
| | | | Example 2 | 89 |
| | | | Example 3 | 89 |
| | | | Example 4 | 95 |
| 7 | F | 32 | Purified water | 63 |
| | | | Example 1 | 91 |
| | | | Example 2 | 90 |
| | | | Example 3 | 88 |
| | | | Example 4 | 97 |
| 8 | M | 30 | Purified water | 66 |
| | | | Example 1 | 95 |
| | | | Example 2 | 96 |
| | | | Example 3 | 94 |
| | | | Example 4 | 98 |
| 9 | M | 38 | Purified water | 51 |
| | | | Example 1 | 81 |
| | | | Example 2 | 82 |
| | | | Example 3 | 80 |
| | | | Example 4 | 90 |
| 10 | F | 45 | Purified water | 59 |
| | | | Example 1 | 92 |
| | | | Example 2 | 93 |
| | | | Example 3 | 91 |
| | | | Example 4 | 96 |

As a result, as can be seen from Table 3, Examples 1 to 4 showed increased moisture index compared to the control group (purified water), and particularly, the effect of increasing the level index according to Example 4 was remarkably excellent.

### Experimental Example 3: Confirmation of an Effect of Skin Barrier Index of the Composition According to the Present Invention

In order to reduce the skin barrier index, the five points were set on the inner part of the arm of 10 testers, and 5 tapes were stripped on the part and then the skin barrier of the part where 5 tapes were stripped using the skin condition measuring device (GPSKIN) The index was measured. Examples 1 to 4 were applied to four of the five points where tape stripping was performed, and after 30 minutes, the skin barrier index was measured using the GPSKIN at the five points and compared with the unapplied points.

TABLE 4

| | | | | Skin |
|---|---|---|---|---|
| | | | | barrier |
| ID | Gender | Age | Sort | index |
| 1 | M | 41 | Stripping point | 76 |
| | | | Example 1 | 84 |
| | | | Example 2 | 82 |
| | | | Example 3 | 82 |
| | | | Example 4 | 86 |
| 2 | F | 44 | Stripping point | 71 |
| | | | Example 1 | 83 |
| | | | Example 2 | 82 |
| | | | Example 3 | 83 |
| | | | Example 4 | 89 |
| 3 | F | 26 | Stripping point | 87 |
| | | | Example 1 | 82 |
| | | | Example 2 | 88 |
| | | | Example 3 | 87 |
| | | | Example 4 | 92 |
| 4 | F | 27 | Stripping point | 76 |
| | | | Example 1 | 90 |
| | | | Example 2 | 88 |
| | | | Example 3 | 86 |
| | | | Example 4 | 92 |
| 5 | F | 26 | Stripping point | 71 |
| | | | Example 1 | 85 |
| | | | Example 2 | 81 |
| | | | Example 3 | 82 |
| | | | Example 4 | 89 |
| 6 | F | 28 | Stripping point | 72 |
| | | | Example 1 | 82 |
| | | | Example 2 | 80 |
| | | | Example 3 | 81 |
| | | | Example 4 | 84 |
| 7 | F | 32 | Stripping point | 51 |
| | | | Example 1 | 76 |
| | | | Example 2 | 78 |
| | | | Example 3 | 75 |
| | | | Example 4 | 85 |
| 8 | M | 30 | Stripping point | 76 |
| | | | Example 1 | 76 |
| | | | Example 2 | 77 |
| | | | Example 3 | 78 |
| | | | Example 4 | 89 |
| 9 | M | 38 | Stripping point | 74 |
| | | | Example 1 | 81 |
| | | | Example 2 | 80 |
| | | | Example 3 | 82 |
| | | | Example 4 | 86 |
| 10 | F | 45 | Stripping point | 72 |
| | | | Example 1 | 78 |
| | | | Example 2 | 79 |
| | | | Example 3 | 76 |
| | | | Example 4 | 82 |

As a result, as may be seen from Table 4, Examples 1 to 4 showed an increased skin barrier index compared to the control group (striping point), and particularly, the skin barrier index according to Example 4 was remarkably high.

### Experimental Example 4: Confirmation of an Effect of Improving Skin Condition of a Cream Formulation Containing the Composition According to the Present Invention

In order to know the effect of improving skin condition of the cream formulation containing the composition according to the present invention, three points were designated on the inside of the arms of 10 testers. In addition, it was classified into a cream added with mixture (test group), and a cream without mixture (control group) and an untreated group, and the TEWL, skin moisture index, and skin barrier index were confirmed.

TABLE 5

| | | | | Skin | Skin | |
|---|---|---|---|---|---|---|
| | | | | barrier | moisture | |
| ID | Gender | Age | | index | index | TEWL |
| 1 | M | 41 | Before Test | 84 | 50 | 6 |
| | | | Control | 76 | 84 | 11 |
| | | | (control group) | | | |
| | | | Test (EQ) | 86 | 100 | 7 |
| 2 | F | 44 | Before Test | 83 | 68 | 5 |
| | | | Control | 71 | 87 | 14 |
| | | | (control group) | | | |
| | | | Test (EQ) | 89 | 99 | 8 |
| 3 | F | 26 | Before Test | 87 | 59 | 5 |
| | | | Control | 88 | 88 | 6 |
| | | | (control group) | | | |
| | | | Test (EQ) | 88 | 99 | 5 |
| 4 | F | 27 | Before Test | 89 | 50 | 3 |
| | | | Control | 76 | 85 | 11 |
| | | | (control group) | | | |
| | | | Test (EQ) | 92 | 99 | 5 |
| 5 | F | 26 | Before Test | 81 | 50 | 7 |
| | | | Control | 71 | 94 | 14 |
| | | | (control group) | | | |
| | | | Test (EQ) | 85 | 100 | 9 |
| 6 | F | 28 | Before Test | 82 | 54 | 8 |
| | | | Control | 72 | 92 | 13 |
| | | | (control group) | | | |
| | | | Test (EQ) | 84 | 100 | 7 |
| 7 | F | 32 | Before Test | 76 | 63 | 7 |
| | | | Control | 51 | 93 | 24 |
| | | | (control group) | | | |
| | | | Test (EQ) | 85 | 100 | 11 |
| 8 | M | 30 | Before Test | 76 | 66 | 6 |
| | | | Control | 76 | 98 | 11 |
| | | | (control group) | | | |
| | | | Test (EQ) | 89 | 99 | 11 |
| 9 | M | 38 | Before Test | 81 | 50 | 8 |
| | | | Control | 74 | 75 | 12 |
| | | | (control group) | | | |
| | | | Test (EQ) | 86 | 100 | 9 |
| 10 | F | 45 | Before Test | 82 | 52 | 6 |
| | | | Control | 66 | 78 | 12 |
| | | | (control group) | | | |
| | | | Test (EQ) | 90 | 100 | 7 |

As a result, it was confirmed that the cream of the preparation example containing fermented rice extracts, pineapple natural sugar ceramide extracts, and the glyceryl glucoside mixture (Example 4) according to the present invention has remarkably superior effects in TEWL relief, the increase of the skin moisture index, and the skin barrier index strengthening, compared to the control group.

As described above, the specific parts of the present invention have been described in detail. Accordingly, it will be apparent to those of ordinary skill in the art that these specific techniques are only preferred embodiments, and the scope of the present invention is not limited thereby. Therefore, it may be seen that the substantial scope of the present invention is defined by the appended claims and their equivalents.

1. A cosmetic composition for improving skin barrier strengthening or moisturizing, comprising a composition comprising fermented rice extracts, pineapple extracts and glyceryl glucoside mixture.

2. The cosmetic composition as claimed in claim 1, wherein the weight ratio of the fermented rice extracts, pineapple extracts and glyceryl glucoside mixture is 1:1:1.

3. The cosmetic composition as claimed in claim 1, wherein the fermented rice extracts contain antioxidants (Thioredoxin) having a concentration of 400 ug/L or more.

4. The cosmetic composition as claimed in claim 1, wherein the pineapple extracts contain a ceramide content of 40 g/L or more.

5. The cosmetic composition as claimed in claim 1, wherein the glyceryl glucoside mixture contains a content of a compound of glycerin and glucose of 60% or more having a purity of 90% or more after chemical bonding between glycerin and glucose.

6. The cosmetic composition as claimed in claim 1, wherein the cosmetic composition is applied to at least one type selected from the group consisting of a patch, mask pack, mask sheet, cream, tonic, ointment, suspension, turbid fluid, paste, lotion, gel, oil, pack, spray, aerosol, mist, foundation, powder, and oil paper.

7. The cosmetic composition as claimed in claim 6, wherein the cosmetic composition is applied or deposited on at least one surface of patch, mask pack or mask sheet.

8. A cosmetic method for controlling condition of a skin of a mammal by improving skin barrier strengthening or moisturizing of stratum corneum except for treatment, comprising:
(a) directly applying the cosmetic composition according to claim 1 to the skin of the mammal, or
(b) contacting or attaching the applied or deposited patch, mask pack, or mask sheet to the skin of the mammal, or
sequentially proceeding the steps (a) and (b).

9. The cosmetic method as claimed in claim 8, further comprising:
(c) removing the patch, mask pack or mask sheet from the skin of the mammal after step (b), and applying the composition for improving skin barrier strengthening or moisturizing to the skin of the mammal.

10. The cosmetic method as claimed in claim 8, wherein the cosmetic composition is used in lotion or cream.
